(19) United States
(12) Patent Application Publication
(10) Pub. No.: US 2025/0231123 A1
(43) Pub. Date: Jul. 17, 2025

(54) METHOD FOR OPERATING AN X-RAY DEVICE, X-RAY DEVICE, COMPUTER PROGRAM, AND ELECTRONICALLY READABLE DATA CARRIER

(71) Applicant: Siemens Healthineers AG, Forchheim (DE)

(72) Inventors: Markus Kowarschik, Erlangen (DE); Marcus Pfister, Bubenreuth (DE); Uwe Fabricius, Erlangen (DE); Philipp Roser, Erlangen (DE)

(21) Appl. No.: 19/012,296

(22) Filed: Jan. 7, 2025

(30) Foreign Application Priority Data
Jan. 17, 2024 (EP) 24152388.5

(51) Int. Cl. G01N 23/083

(52) U.S. Cl. CPC G01N 23/083; G01N 2223/076

(57) ABSTRACT

A method for operating an x-ray device includes: recording a series of x-ray images of a recording area, (e.g., for monitoring the recording area); and determining a series of output images to be output from the x-ray images. When the x-ray images are recorded, there is a repeated change between at least two sets of recording parameters that leads to the different representation of at least two different objects of the recording area in the x-ray images. Further, the output images are determined in each case at least from one set of x-ray images that includes x-ray images recorded with at least two of the at least two sets of recording parameters.

The present patent document claims the benefit of European Patent Application No. 24152388.5, filed Jan. 17, 2024, which is hereby incorporated by reference in its entirety.

### TECHNICAL FIELD

The disclosure relates to a method for operating an x-ray device, wherein the method includes recording a series of x-ray images of a recording area, in particular for monitoring the recording area, and determining a series of output images to be output from the x-ray images. The disclosure also relates to an x-ray device, a computer program, and an electronically readable data carrier.

### BACKGROUND

In x-ray applications, it is known to record a series of x-ray images of a recording area over a period, in particular, for monitoring purposes. To optimize the representation, further image processing acts may be carried out before the x-ray image or an image derived from the x-ray image is output as an output image. For example, an intervention on an object under examination and/or a change may be monitored as a process. One or more objects in the recording area may play a role in such monitoring. Low x-ray doses may be used in medical technology applications, in particular when the object under examination is a patient. This type of monitoring, for example, during medical interventions, is also known as fluoroscopic monitoring or fluoroscopy. Such monitored interventions are also known as image-guided interventions. For example, image-guided minimally invasive interventions are known in medical technology.

An x-ray device with a C-arm may be used as the x-ray device, on which an x-ray emitter arrangement and an x-ray detector are arranged opposite one another. As such C-arm x-ray devices may be used for monitoring interventions on vessels in the vascular system, they are also known as angiography systems.

When monitoring processes, in certain examples, at least one object in the examination area is recognizable or visible in the output images. This may be a feature of the object under examination, (e.g., an anatomical feature of a patient), or an instrument or an active substance, (e.g., a tool in the case of workpieces). In minimally invasive interventions on a patient, interventional objects may be relevant, with these including, for example, medical instruments (e.g., needles, catheters, guidewires), implants (e.g., stents, coils, spirals), and active substances (e.g., contrast media, embolization agents).

In order to provide the visibility of at least one relevant object, it is known to select recording parameters, in particular of the x-ray emitter arrangement, but also of the image generation chain, in such a way that the material properties of the relevant object are taken into account and thus the representation/visibility may be improved, in particular optimized, during monitoring, in the x-ray images and thus output images. Recording parameters of the x-ray emitter arrangement include, for example, the tube voltage and the tube current of an x-ray tube, the pulse width, the pulse length or shot time, filter elements to be used for the x-ray field, and the like. In addition, object-specific image processing algorithms are known that may improve the visibility or representation of an object, for example, by taking into account its geometry (e.g., shape) and/or the speed at which the object moves in the recording area. Furthermore, image processing measures, in particular image processing measures prescribed in the image generation chain, may also be aimed at movement correction and the like. For example, fluoroscopic monitoring of a beating heart requires a different temporal smoothing algorithm or a differently parameterized smoothing algorithm than fluoroscopic imaging of the brain.

However, this may lead to the problem that several objects with different material properties are relevant for x-ray imaging. This makes it difficult to select suitable recording parameters in order to visualize all these objects sufficiently well. In known solutions, this problem may be left to the user, who manually selects a suitable system configuration, e.g., a suitable set of recording parameters. As a consequence, this leads to sub-optimal representation/visibility of at least some of the relevant objects. This also occurs if a set of recording parameters is selected that is matched to a relevant object of a certain object class, after which other objects may be poorly visible or even not visible at all.

In particular, various processes are known in the field of medical technology in which combinations of objects are relevant in the representation, for example, including anatomical features and/or combinations of interventional objects. For example, in what is known as stent-assisted coiling, both a stent and a coil, as well as any associated instruments, are monitored. In stroke therapy, for example, different types of catheters or general instruments are used, such as an aspiration catheter and a stent retriever. Other types of instruments with special material properties that are used include, for example, balloons for cavity closures and the like.

In the prior art, solutions have already been proposed to better highlight individual relevant objects in output images during monitoring using x-ray imaging. For example, a method known as “ClearStent” has become known that uses image data from the past to improve the recognizability of an object in current output images.

### SUMMARY AND DESCRIPTION

The object of the disclosure is to improve the visibility of several such objects when recording a series of x-ray images of a recording area with objects of different material properties with regard to x-ray imaging.

According to the disclosure, this object is achieved by a method, in particular a computer-implemented method, an x-ray device, a computer program, and an electronically readable data carrier as disclosed herein. The scope of the present disclosure is defined solely by the appended claims and is not affected to any degree by the statements within this summary. The present embodiments may obviate one or more of the drawbacks or limitations in the related art.

In certain methods, when the x-ray images are recorded, there is a repeated change between at least two sets of recording parameters which lead to the different representation of at least two different objects of the recording area in the x-ray images, and the output images are determined in each case at least from one set of x-ray images that includes x-ray images recorded with at least two of the at least two sets of recording parameters.

It is therefore proposed to work with several sets of recording parameters during a recording process, e.g., the recording of the series of x-ray images. In particular, each set of recording parameters is used several times. With two sets of recording parameters, this means that during the recording of the series of x-ray images, the system switches from the first set of recording parameters to the second set of recording parameters not just once, but also switches from the second set of recording parameters back to the first set of recording parameters, which happens repeatedly. This means that different sets of recording parameters are used alternately over time, from whose x-ray images, when viewed together, output images may be determined that allow at least one of the different objects to be recognized more easily than when only one of the sets of recording parameters is used.

This utilizes the fact that methods have now been proposed in the prior art that allow extremely fast switching between recording parameters, particularly with regard to the x-ray emitter arrangement. For example, it may be possible to change the set of recording parameters 60 to 100 times per second. Thus, it is a concept of the present disclosure to quickly switch between different sets of recording parameters, which depict different objects differently in the x-ray images, and to generate a merged output image with increased visibility for all relevant objects. An enhanced, improved recorded image of relevant objects is generated for the user on the basis of the x-ray images, which were recorded with different sets of recording parameters.

In this way, it is therefore possible to achieve a simultaneous improvement in the visibility of these multiple objects, even if they have different properties in terms of x-ray imaging.

In particular, the recording area includes at least part of an object under examination. This may be a workpiece or the like, for which, for example, machining processes or other processes, such as testing processes, for example, may be monitored. However, the method is particularly advantageous in the field of medical technology. In this case, the object under examination may be a patient. The technique described here may be used to monitor physiological processes, but also for imaging during, in particular, minimally invasive interventions.

The procedure may be applied particularly advantageously to fluoroscopic monitoring (fluoroscopy), in particular in medical technology. The present method deals solely with the imaging of recording areas by controlling the x-ray device and processing the resulting x-ray images. In other words, the disclosure relates to an imaging method that does not involve or necessarily require any diagnostic, surgical, or therapeutic measures.

The x-ray device may be an x-ray device with a C-arm, on which the x-ray emitter arrangement and the x-ray detector are mounted opposite one another. A C-arm makes it possible to flexibly set different recording geometries and thus select a projection direction that allows ideal monitoring of the recording area. In certain examples, the method may be carried out by a control device of the x-ray device, wherein the control device is discussed in more detail below.

The sets of recording parameters may differ at least in pairs in at least one recording parameter of the x-ray emitter arrangement and/or in at least one recording parameter of the image generation chain. Here, the recording parameters of the x-ray emitter arrangement may be selected from the group including a tube voltage and/or a tube current and/or a pulse width of an x-ray tube of the x-ray emitter arrangement, and/or a filter element of the x-ray emitter arrangement to be used and/or a focus size of a focal point of the x-ray emitter arrangement and/or a pulse length of the x-ray pulse. The tube voltage plays a particularly important role here. If, for example, a fine-meshed object made of iron, such as a stent, is compared with a coarser-structured object made of platinum, such as a coil in stent coiling, if there is a good representation of the structure of the first object due to a first set of recording parameters, that of the second object may not be recognizable. An adjustment of the tube voltage for a second set of recording parameters may already enable improved recognizability of the structure of the second object mentioned as an example (possibly in such a way that the first object may no longer be recognized). Adjusting the filtering of the x-rays, for example, by selecting the filter material and/or the filter geometry, may also improve the representability of certain objects using x-ray imaging. The focus size, the pulse width, the pulse length (or, in the case of continuous illumination, the shot time), and other parameters may also be varied within the scope of the disclosure.

However, the procedure described here also allows the recording parameters of the image generation chain to be adapted in order to enable object-specific preparation of the x-ray image from the raw data. With regard to the recording parameter of the image generation chain, the parameter may be selected from the group including a noise treatment parameter and/or a filtering parameter and/or a resolution parameter, in particular a spatial frequency, and/or a correction parameter, in particular with regard to artifact correction and/or movement correction. In particular, knowledge about the objects that may be particularly recognizable may be incorporated here. For example, a spatial frequency used in the image processing chain may be selected according to a spatial frequency of the structures of at least one of the objects. Information on the movement of at least one object may also be received, for example, with regard to movement correction/movement smoothing. Recording parameters of the image generation chain may also determine which image processing functions are to be applied, so that, for example, image processing functions that make certain objects more visible may be selected.

In a specific embodiment, the sets of recording parameters may be used varyingly for one x-ray image at a time according to a predetermined sequence, alternating in the case of two sets of recording parameters. In particular, each set of recording parameters is used for one x-ray image here. If two sets of recording parameters are used, one x-ray image may therefore be taken with the first set of recording parameters, and one x-ray image with the second set of recording parameters, alternately. A fixed, repetitive sequence may also be used for more than two sets of recording parameters, for example (first set of recording parameters, second set of recording parameters, third set of recording parameters).

In an expedient development, it may be envisaged that the frequency of changing the sets of recording parameters is higher than the frequency for determining the output images to be output, in particular in such a way that exactly one x-ray image is recorded for each of the sets of recording parameters for the determination of each output image. In other words, the sets of recording parameters for the various relevant objects may be changed at a higher temporal frequency than the targeted visualization frequency. This has the advantage that the user receives a better visualization of the recording area without any loss of visualization frequency. For example, two sets of recording parameters may be provided for the x-ray images to be recorded at twice the frequency at which the output images are provided.

A variant of the disclosure may also provide that the sets of recording parameters are changed more quickly than the human eye is able to resolve image changes, and that the resulting x-ray images are output as an output image at the frequency at which they are recorded. In this way, the image impressions and thus the output image are implicitly composed by the human brain.

One expedient embodiment provides that, for the determination of the output image from the x-ray images of different sets of recording parameters: (1) an, in particular weighted, at least partial overlaying of at least two of the x-ray images takes place; and/or (2) the x-ray image data of at least one x-ray image in a region, determined in particular by segmenting an object in the same or another of the x-ray images, is replaced by x-ray image data of another of the x-ray images and/or replacement image data representing in particular the segmented object; and/or (3) the output image has at least a partial pixel- and/or patch-based composition.

The extended, merged output image may be determined in different ways, for example, in easily realizable embodiments, by calculating a weighted mean value across the x-ray images. In certain examples, image areas of a segmented object may be overlaid or replaced with an image of the object (e.g., replacement image data). A region-by-region replacement of x-ray image data of an x-ray image with x-ray image data of another x-ray image, or replacement data, is particularly useful if the representation deficiency of an object when using a set of recording parameters is due to the fact that its structure, in particular its internal structure, is not imaged or is not imaged with sufficient accuracy. However, if this is the case in another x-ray image when using a different set of recording parameters its x-ray image data may be used. For example, when using higher x-ray doses for the representation of finer structures, an effect may be observed where strongly attenuating objects are instead imaged due to their structure as a “weakening spot” that may be segmented (here in particular using the same x-ray image in which the replacement is to be made), whereupon x-ray image data from another x-ray image may be used. Finally, it is also possible to compose the output image pixel by pixel or patch by patch, in particular also based on segmentation results and/or other region of interest information.

Within the scope of the present disclosure, at least two sets of recording parameters may be predefined, (e.g., independently of the object), at least for one monitoring task. In other words, at least two defined sets of recording parameters may be used, independently of the particular imaging task and the objects present in the recording area. The sets of recording parameters are selected in such a way that they each cover a wide range of materials, spatial frequencies and the like, so that in their combination they may capture as many objects as possible with acceptable quality. This makes it particularly easy to implement the concept proposed according to the disclosure, while still allowing an improvement compared to the use of just a single set of recording parameters.

However, if each set of recording parameters is assigned to an object class of a selected object present in the recording area and, in particular, is configured to the object class, the visibility of objects of the object class may be higher in x-ray images recorded with the set of recording parameters associated with the object class than in x-ray images recorded with sets of recording parameters associated with other object classes. In this case, relevant objects present in the recording area are selected, the sets of recording parameters being specifically selected for high-quality visualization of object classes of these selected objects. In this way, the output image may be optimized to visualize the selected objects.

In this case, a visibility measure, as is generally known in the prior art, may be used to assess the visibility in an x-ray image, in particular to obtain optimized sets of recording parameters for certain object classes of selected objects, as is already known in the prior art for highlighting individual objects. Visibility measures may describe the contrast and/or the recognizability of the structure of objects in the object classes.

Here, the object classes may ultimately be defined in as much detail as desired. For example, it is conceivable to define the object class by an object feature, (e.g., a material or a material property), of the object. Furthermore, the definition of object classes may include the fineness of the structuring of the object that is to be made visible.

In this embodiment, in other words, the at least two sets of recording parameters may be specifically selected so that at least two selected objects in the recording area, which cannot be recorded in sufficient quality, e.g., sufficient visibility, with only one set of recording parameters, may be represented in the output image in a sufficiently (e.g., clearly) visible manner, especially with regard to their structure. The selected objects may concern a process to be monitored or be actors in it. In the example of monitoring a minimally invasive procedure, a selected object may be chosen, for example, from the group including an anatomical feature, a medical instrument, an implant, an active substance, or an aid.

The sets of recording parameters for the various object classes and, where necessary, the assignment of objects to object classes, may be stored in at least one database. In-particular, as already explained above, the sets of recording parameters may be optimized for the representation of the respective at least one object class.

Here, it is first of all conceivable that the selected objects (and thus object classes) are selected based on user input. For example, known objects may be represented graphically and/or as text in a user interface, so that the user may manually select relevant objects that they wish to see clearly visible, in particular optimally visible, in the output image. Here, the list of known objects may similarly be completed manually or may be completed at least partially automatically. For example, a process that has been carried out and/or the object under examination may already result in an assigned quantity of objects located in the recording area. Known objects may also have been detected by a sensory device or mechanism, and, for example, also by a targeted labeling for identification. For example, RFID chips, bar codes, or the like may be read from objects used in the recording area. Algorithms may also be used for pattern recognition. During medical procedures on a patient as an object under examination, the type of procedure may be derived from previously used medical instruments, implants, active substances and/or aids, as may anatomical features. Missing information, (e.g., materials, models, and the like), as well as missing objects, may be added as described, either by a sensory device and/or by user input.

In a particularly advantageous development, the objects may be selected at least partially automatically based on a prioritization of known objects, during which all known objects are given a priority value. The determination of the known objects may take place as described above for the user selection of known objects from lists. Expediently, here, objects that are assigned to the same object class with regard to the sets of recording parameters, are considered as a common known object, so that the same object class is not selected twice. This may also be formulated so that prioritization for object classes is based on the largest priority value of the objects belonging to it (which then defines the selected object).

Prioritization may be performed in such a way that all known objects are assigned a priority value. A number of the highest-prioritized known objects from various object classes are automatically selected. The prioritization may be fixed or changeable over time, which is discussed in more detail below. The number of objects to be selected and thus object classes may be constant or predefined for a process to be monitored. For example, a number of objects to be selected, for example two, may be specified, so that this number of highly-prioritized objects is selected. However, the number may also vary, for example, if a threshold value of the priority is used additionally or alternatively. It is also possible to have (additional) time intervals in which only one selected object exists, and thus only one set of recording parameters is used.

The automatic selection of at least one object for particularly visible representation during x-ray monitoring may also be advantageous independently of the change of sets of recording parameters. In other words, a method of operating an x-ray device is conceivable, wherein the x-ray device has: an x-ray emitter arrangement; an x-ray detector for receiving x-ray radiation of an x-ray radiation field emitted by the x-ray emitter arrangement; an image generation chain for determining an x-ray image of a recording area from raw data recorded with the x-ray detector; and an output device for outputting an output image including or determined from the x-ray image. The method includes recording a series of x-ray images of a recording area, in particular for monitoring the recording area, and determining a series of output images to be output from the x-ray images. The method further includes selecting at least one object based on a prioritization of known objects present in the recording area, in which all known objects are assigned a priority value. Further, the method includes, for each selected object, selecting at least one set of recording parameters assigned to an object class of the selected object, in particular one that is matched to it, the visibility of objects of the object class being higher in x-ray images recorded with the set of recording parameters associated with the object class than in x-ray images recorded with sets of recording parameters associated with other object classes. The method further includes recording x-ray images using the at least one selected set of recording parameters.

Here, all the preceding and following statements may also be applied to such a conceivable procedure. In particular, such a conceivable method also allows for embodiments that only ever refer to a selected object and are aimed at achieving an excellent representation of this most relevant object.

In the conceivable method, several relevant, (e.g., selected), objects may also be represented more clearly by dividing them into time segments in which the x-ray image used as the output image is recorded with one of the assigned sets of recording parameters in each case. If the time segments are the same length, this is referred to as regular time slicing. Also possible, however, is an irregular time slicing, in which the length of the time segments may for example be selected according to the priority values. In this way, a highly relevant selected object is represented in an optimized way in a relatively large proportion of the x-ray images, while the at least one selected object that requires less attention is represented in an optimized way less frequently.

Automatic prioritization and selection of objects, for whose visibility the output image is to be optimized, relieves the user of a task, and also allows for dynamic adjustment if, for example, other objects become more relevant than previously represented objects in the course of a monitored process. An embodiment with automatic selection of objects (and thus sets of recording parameters) is particularly advantageous when the user is involved in the process, for example, in the case of an intervention on the object under examination. This means that the user does not have to break their concentration and may still receive the information they actually need by looking at the output image. In particular, the objects that are currently being moved and/or interacted with may be selected during an intervention, for example. In particular, with selection updating, as discussed in more detail below, (e.g., dynamically variable selection of objects), it is also possible to specifically select those known objects that require the user's attention.

Regardless of the sets of recording parameters, objects, or object classes used in the monitoring of a particular process, a lower priority value may be assigned to objects that are sufficiently visible with all sets of recording parameters than to objects that are less visible or not visible at all when some sets of recording parameters are used. This improves/provides the visibility of poorly visible objects. If, for example, an object of simple, extended structure made of metal is present in the recording area, it may be assumed that this object is sufficiently visible regardless of the set of recording parameters, so that a high prioritization is not necessary here. Exemplary embodiments are also conceivable in which known objects may be effectively excluded for prioritization, for example, those whose priority value may be set to the lowest possible priority. This applies not only to objects that are sufficiently visible anyway, but also to familiar objects for which it is known that their representation is not relevant due to the nature of the process to be monitored. This applies, for example, to minimally invasive medical interventions for anatomical features that are not affected and/or anatomical features that are added to the output image as an overlay anyway, for example, from a prior image data set.

An exclusion of known objects or a minimal prioritization for known objects may also be carried out on the basis of an eye tracking of a user. For example, only those objects that are located in a region that the user is viewing may therefore be used as evaluable objects. This supports the user who is looking in that region by representing the relevant objects so they are particularly easy to recognize.

A development of the present disclosure may provide that the determination of the priority value of the prioritization for at least one known object is carried out taking into account dynamic information that describes a current dynamic of the object. Here, dynamic information may relate to any kind of conceivable change, for example rigid-body movements (rotations, translations) as well as changes in shape, expansion, and the like. The consideration of dynamic information is based on the idea that known objects with a high degree of dynamism may also play a relevant role in the process, as for example for moving instruments, spreading and/or consuming active substances, features moved by external influence and the like. In other words, a high dynamic indicates high relevance for many processes. While for dynamic information, various sources are conceivable, (e.g., tracking sensors, positioning systems, and the like), in certain examples, the dynamic information may be determined at least partially from x-ray images.

It may be provided that, for the determination of the dynamic information, at least one x-ray image showing the respective known object, in particular a time series of x-ray images, is evaluated by a, in particular trained, dynamic determination function. In this way, for example, an instrument that a user is moving may be identified, or an active substance that is currently active may be recognized. In this case, the dynamic determination functions determining the movement of objects or tracking objects are already basically known in the prior art, for example, in the tracking of objects in road traffic and the like. Corresponding dynamic determination functions may also be used in the context of the present disclosure to automatically determine the dynamic information in a simple way.

The dynamic determination function may be trained by machine learning. In certain examples, a trained function maps cognitive functions that humans associate with other human brains. Through training based on training data (machine learning) the trained function is able to adapt to new situations and to detect and extrapolate patterns.

The parameters of a trained function may be adjusted by training. In particular, supervised learning, semi-supervised learning, unsupervised learning, reinforcement learning, and/or active learning may be used. Furthermore, representation learning (also known as feature learning) may be used. The parameters of the trained function may in particular be adapted iteratively through a plurality of training acts.

A trained function may including a neural network, a Support Vector Machine (SVM), a decision tree, and/or a Bayes network, and/or the trained function may be based on k-means-clustering, Q-learning, genetic algorithms, and/or mapping rules. In particular, a neural network may be a deep neural network, a convolutional neural network (CNN), or a deep CNN. Furthermore, the neural network may be an adversarial network, a deep adversarial network, and/or a generative adversarial network (GAN).

In the present case of image processing, a CNN is particularly suitable for the dynamic determination function.

In particular, when the method is used in the field of medical technology, the recording area itself may be subject, at least in part, to a fundamental movement, (e.g., a physiological movement like breathing and/or heartbeat). In such cases, it may be expedient to provide that, in the case of a recording area subject to physiological movement, this is taken into account when determining the dynamic information, in particular, it is factored out. For this purpose, a movement correction and/or a movement separation may be performed.

Various specific approaches for determining dynamic information using the dynamic determination function may be used in the context of this embodiment. Thus, in a first embodiment, for example, it may be provided that artificial intelligence, for example, also a trained portion of the dynamic determination function, is used to recognize (known) objects in the x-ray images of the time series. On this basis, known methods of movement detection or object tracking may be used, for example, optical flow methods. Also conceivable, however, is an embodiment, in which the trained dynamic determination function receives as input data the x-ray images of the time series, annotated if necessary, so that it may provide as output data (in particular object-specific) movement fields, and/or heat maps. Also possible is an embodiment, in which a trained dynamic determination function solely on the basis of the time series of x-ray images already provides the dynamic information mapped to the objects as output data.

In a specific embodiment, at least a speed and/or a speed curve of the object may be determined as dynamic information. Speed may, for example, be determined in pixel pitches between individual x-ray images per unit of time. In particular, in the determination of speeds and/or speed curves, it is expedient to separate and/or correct physiological movements. In this context, artificial intelligence may also be advantageously used, particularly with regard to the separation of movements, for example, to separate heart movement and/or breathing movement from other movements, such as those that affect the specific process.

When determining the dynamic information for a fluidic object, for example, an embolization agent bolus or a contrast medium bolus, a propagation front of the fluidic object may be tracked and/or a gradient of the concentration of the fluidic object may be determined. It is therefore also possible to determine meaningful dynamic information for fluidic objects, for which rigid movements play little or no role, providing clues as to their relevance. For example, after the administration of an active substance, such as a contrast medium or an embolization agent, there is initially a phase of high dynamics with significant changes, which may be detected accordingly via the movement of the bolus front and/or concentration changes.

Embodiments of the present disclosure may also provide that the determination of the priority value of the prioritization for at least one known object takes place considering workflow information relating to a monitored process. In particular, the workflow information may be determined by a trained workflow determination function.

Detection of a current workflow act based on artificial intelligence may therefore also take place, as has already been proposed in the prior art, for example, to provide context-sensitive help. Such a determination of the workflow information may use input data from various sensors in the process environment, in particular the intervention environment, and also the x-ray imaging itself, to determine the current actual position in a workflow describing the process. From this, it may in turn be inferred which objects are of direct relevance and which objects are less relevant to the current situation. In particular, based on the workflow information some objects, in particular interventional objects, may be excluded from the quantity of known objects or their priority value selected to reflect the lowest priority. If a current workflow act involves, for example, the placement of an implant or aid in the recording area while an instrument with another aid is in a waiting position in the recording area, the first-mentioned implant or aid and its instrument may be prioritized higher than the other instrument with the other aid.

In this context, with regard to the sets of recording parameters to be changed, it is quite conceivable for the priority values to also be included in the change process, for example, in such a way that a weighting of the use of the sets of recording parameters takes place according to the priority values. If, for example, a first selected object is prioritized twice as highly as a second selected object, the set of recording parameters assigned to the first selected object (or its object class) may be used twice as often as the set of recording parameters assigned to the second selected object (or its object class). However, this is only really useful when the priority values differ greatly.

In a particularly expedient embodiment with automatic selection of the objects/object classes, dynamic updating is provided for. This means that it may be provided that an update of the known objects and/or priority values and of the selected objects takes place at regular intervals, in particular after each nth x-ray image and/or output image, or an update takes place on an event basis.

In a first variant, a periodic update is provided for here. After recording each nth x-ray image or output image, an update may take place, e.g., after each determination of an output image. This means that it is possible to react to a change in priorities in a timely manner.

It is also possible to adaptively check for switching when an event occurs. Specifically, the occurrence of a user-defined trigger may be used as an event. In this case, for example, by a user interface a user may define one or more triggers, upon the occurrence of which a check is made whether an update to the selected objects is necessary. The occurrence of the event is determined using the dynamic information and/or the, or further, workflow information. Thus, for example, in the event of a change to the workflow situation, for example, in the event of a change to another workflow act, a check is made whether an update to the selected objects is necessary. Additionally, the dynamic information may indicate which objects are particularly active. If there is a change here, so if another known object becomes active, while a previously active object ceases to be so, an update may likewise be triggered.

It may further be provided that the presence of an externally provided trigger signal is used as an event. An injector, (e.g., for a contrast medium), may send a trigger signal to the x-ray device that a contrast medium has been administered, whereupon the contrast medium may be given a higher priority and the recording parameters may be selected in such a way that the contrast medium is represented as well as possible in the output image. In another example, when using a robot device, (e.g., endovascularly or percutaneously), the robot device may inform the x-ray device about the specific intervention object being moved. This may then also be given a higher priority.

A development of the present disclosure may provide that to increase the visibility of at least one known, in particular selected, object to be highlighted using the image information on the object in past x-ray images, object appearance information, in particular statistical, is determined and a current x-ray image in particular recorded with the set of recording parameters assigned to the object to be highlighted. Additionally, or alternatively, the current output image is modified using the object appearance information, in particular by inserting the object appearance information in an image area of the object to be highlighted. Such a procedure is expedient in particular if the object is difficult to recognize, for example, noisy or the like. In this way, an improved, clearly visible representation of the object is determined with the addition of past image information of the object and used in the output image. Thus, for example, in the case of a noisy object, significantly improved contrasting may be achieved. Here, the object appearance information may also be determined using the image information on the object in the current x-ray image, thus as much information as possible is included.

Specifically, it may be provided that the image information of the various x-ray images is determined for determination of the object appearance information. In other words, an improvement therefore takes place in the visibility of the object based on time averaging of the image information, in order to extract and highlight object details and to reduce (statistical) noise.

In the event of, for example, a physiological movement in the recording area, it may be necessary for determination of the object appearance information, to register the x-ray images with respect to each other, in particular based on at least one feature of the object. Therefore, in particular in the event of a movement of the object to be highlighted, the past x-ray images and, where used, the current x-ray image, may be registered with respect to each other.

Apart from the method, the disclosure also concerns an x-ray device. The x-ray device includes an x-ray emitter arrangement, an x-ray detector for receiving x-ray radiation of an x-ray radiation field emitted by the x-ray emitter arrangement, an image generation chain for determining an x-ray image of a recording area from raw data recorded with the x-ray detector, an output device for outputting an output image including or determined from the x-ray image, and a control device. The control device includes a recording unit for recording a series of x-ray images of a recording area, in particular for monitoring the recording area, and a determination unit for determining a series of output images to be output from the x-ray images.

Here, the recording unit is configured, when the x-ray images are recorded, to repeatedly change between at least two sets of recording parameters that lead to the different representation of at least two different objects of the recording area in the x-ray images. Further, the determination unit is configured to determine the output images in each case at least from one set of x-ray images that includes x-ray images recorded with at least two of the at least two sets of recording parameters.

The control device has at least one processor and at least one storage device. It includes functional units, which may be formed of hardware and/or software and in particular may also execute acts of the method as described herein. The recording unit controls the x-ray emitter arrangement, the x-ray detector, and the image generation chain for recording x-ray images according to the sets of recording parameters. The determination unit includes suitable image processing devices or mechanisms for generating output images. Further functional units for executing further, optional acts of the method are likewise conceivable. In particular, a selection unit for selecting the objects, in particular based on the prioritization, may be provided. The selection unit specifies to the recording unit for the selected objects or their object classes the sets of recording parameters to be used.

The x-ray device may involve a C-arm x-ray device, e.g., as part of an angiography environment.

A computer program may be loaded directly into a storage device of a control device of an x-ray device and has program code such that when the computer program is executed on the control device this causes the control device to carry out the method. The computer program may be stored on an electronically readable data carrier according to the disclosure, which therefore includes control information stored thereon, which includes at least a computer program and is designed such that when the data carrier is used in a control device of an x-ray device this is embodied to perform a method as described herein. The data carrier is, in particular, a non-transient data carrier, for example, a CD-ROM.

### BRIEF DESCRIPTION OF THE DRAWINGS

Further advantages and details of the present disclosure are specified in the exemplary embodiments described below and in the drawings.

FIG. 1 depicts a flowchart of an embodiment of the method.

FIG. 2 schematically depicts an example of a first x-ray image recorded with a first set of recording parameters.

FIG. 3 schematically depicts an example of a second x-ray image recorded with a second set of recording parameters.

FIG. 4 schematically depicts an example of an output image determined from the first and the second x-ray image.

FIG. 5 depicts an example of a schematic diagram of an x-ray device.

FIG. 6 depicts an example of the functional structure of a control device of the x-ray device.

### DETAILED DESCRIPTION

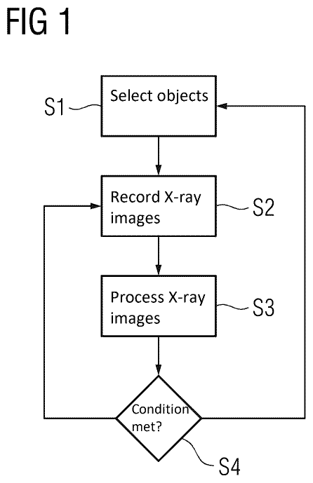

FIG. 1 shows a flowchart of an embodiment of the method. Here, as an exemplary explanation, the image monitoring of a minimally invasive, stent-assisted coiling intervention in the region of a brain aneurysm is discussed, without this calling into question the general applicability of the method.

In the embodiment represented in FIG. 1, a process in a recording area is being monitored by x-ray imaging. Here, relevant objects for a user are to be represented with optimized visibility. To this end, in act S1, the objects or object classes are selected from a list of known objects, for which the x-ray imaging is to take place in an optimized manner. To this end, a prioritization is used.

The list of known objects in the recording area or the occurring object classes is, for example, managed in a selection unit of a control device of the x-ray device used. The presence of objects in the recording area and thus the list of known objects may be derived from a variety of information. Thus, for example, the actual process to be monitored provides clear indications of which objects are already present in the recording area, for example, of the object under examination, and which objects, in relation to an intervention, for example, interventional objects, are also present there for the process. This may be further specified as part of a specific workflow that is followed. For example, by an in particular trained workflow determination function, workflow information may be determined that, for example, a current workflow act may display. In turn, this may be assigned objects necessary for the purpose in the recording area. The workflow information may also be used with regard to the determination of priority values for the prioritization.

The presence of objects in the recording area may also be determined from a user input and/or by a sensory device. Objects present in the recording area may also be inferred from x-ray images, for example, by detection of the objects in the x-ray images by suitable, in particular trained, detection functions, as are known in the prior art.

Here, in a storage device of the control device of the x-ray device a database is also stored, which optionally assigns object classes to objects, but, in any case assigns sets of recording parameters to object classes, which include recording parameters matched to objects of the object classes for an x-ray emitter arrangement and an image generation chain of the x-ray device. This means that the recording parameters are selected taking into account the relevant properties for x-ray imaging of the objects of the object classes, for example, their material and their structure (for example occurring spatial frequencies), such that the objects of the object classes are represented with greater visibility with this assigned set of recording parameters than with sets of recording parameters assigned to other object classes. In particular, the recording parameters are selected for optimization of the visibility of the objects of the object classes.

If a number of objects occur under the known objects, which are to be assigned to the same such object class, these are dealt with as one known object. In other words, the object classes are ultimately prioritized and selected. Here, the highest priority value of the known objects of the object classes for the object classes or the amalgamated overall object is used, whereby all known objects of the same object classes are counted as a single selectable known object with the maximum priority value of the individual known objects. Thus, each object class is selected only once, in that only its most representative object, thus the one with the highest priority, is selectable. The selection process may be designed, however, in such a way that in the event of a plurality of objects to be selected these cannot belong to the same object class.

Here, certain objects may also be assigned the lowest possible priority value, or they may even be fully removed from consideration. This may apply to objects that are otherwise recorded in the output image (for example, anatomical features, which are overlaid from a prior image data set), but also to those for which, for example, it is known from the workflow information that they are not relevant for the current workflow situation. In addition, it is conceivable to track the view of the user (e.g., eye-tracking) and to only provide objects in the field of view of the user with a priority value exceeding the minimum.

In certain examples, for determination of the priority values for known objects, at least the already mentioned workflow information and dynamic information on the respective known object is used. The dynamic information describes the dynamics of the object, wherein higher dynamics are assumed to be of greater relevance. The workflow information also describes which known objects are relevant in the current workflow situation and how. In this case, the dynamic information is determined from a time series of x-ray images, which display the corresponding object, the time series of x-ray images, in particular already annotated, being used as input data of, in particular, a trained, dynamic determination function. In the event of physiological movements, these are in particular corrected or separated as part of movement separation. The dynamic information may, in particular for solid objects, include a speed or a speed curve. For fluidic objects, for example, a bolus, the movement of a bolus front and/or a change in concentration may be determined as dynamic information. If, for example, in a stent-assisted coiling, the medical instrument, in particular the catheter, is moved with the coil, this suggests that the coil is to be positioned and that therefore this and possibly the corresponding catheter constitute relevant objects. A correspondingly high priority value is selected. Other information may also be included in the determination of the priority values, for example, as described above, in order to determine known objects to be excluded.

In act S1, the known objects in the recording area may then be selected that have the highest priority value, and thus the highest priority (and belong to different object classes). Here, a fixed number of objects to be selected may be provided for, (e.g., two), but varying numbers, (e.g., in the case of threshold values for the priority value for selection), may also result.

In act S2, x-ray images are then recorded, using the sets of recording parameters in the database, which are assigned to the selected objects/object classes. Here, in the present exemplary embodiment, in a predetermined sequence, in each case, exactly one x-ray image is recorded with each set of recording parameters through corresponding control of the x-ray emitter arrangement, the x-ray detector and the image generation chain. For example, in the case of two selected objects x-ray images are recorded alternately with the sets of recording parameters.

Thus, in act S2, switching between sets of recording parameters takes place at higher speed. In the present embodiment, the recording parameters of the sets of recording parameters include both sets of recording parameters for the x-ray emitter arrangement that differ in pairs and recording parameters for the image generation chain that differ in pairs. Recording parameters, which are selected differently for different object classes, may include, for the x-ray emitter arrangement, a tube voltage and/or a tube current of an x-ray tube of the x-ray emitter arrangement and/or a filter element to be used of the x-ray emitter arrangement and/or a focus size of a focus point of the x-ray emitter arrangement. Additionally, or alternatively, for the image generation chain, the recording parameters may include a noise treatment parameter and/or a filtering parameter and/or a resolution parameter and/or correction parameter, in particular with regard to an artifact correction and/or a movement correction. Here, switching between recordings takes place at a frequency that is higher than a frequency at which in act S3 output images are determined and output. For example, x-ray images may be recorded at a rate of 60 to 100 fps, (e.g., 80 fps), switching for each x-ray image (each frame) between the two sets of recording parameters available. Here, known techniques for fast switching may be used for other purposes, in particular with regard to the x-ray emitter arrangement.

Certain embodiments are conceivable in which switching between sets of recording parameters is so fast that with direct output of the x-ray images to an output device the humanly perceptible change frequency is exceeded and as a result in the mind of the observer the output image implicitly results from a combination of the x-ray images of various sets of recording parameters.

In the present case, in the control device, a determination unit is present that processes a set of x-ray images in act S3, which for each set of recording parameters includes at least one x-ray image (e.g., exactly one x-ray image) that has been recorded with this set of recording parameters, into an output image, in which all selected objects are identifiable, and outputs the output image.

In act S3, from the object class x-ray images, which may be considered as specific x-ray images for a selected object, an improved output image for the user with regard to all selected objects is generated. This may take place by in particular weighted, pixel by pixel averaging of the individual x-ray images, overlaying segmented selected objects on one of the x-ray images, patch-based generation based on regions of interest, and/or pixel-based generation. In particular, x-ray image data of one of the x-ray images may be replaced region by region by x-ray image data of another of the x-ray images.

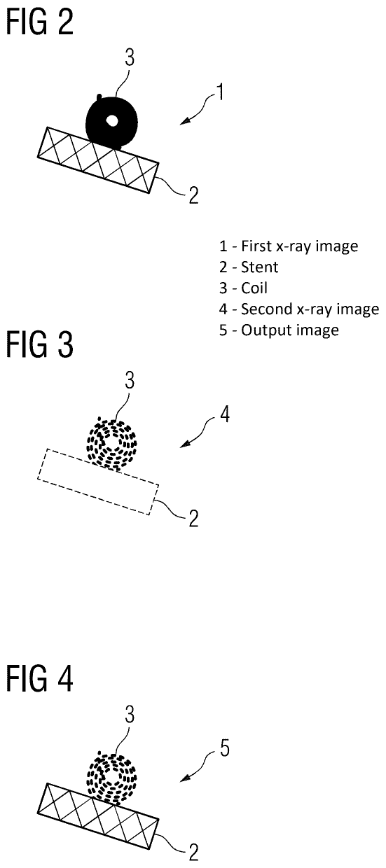

This is represented by way of example for a special application case in FIGS. 2 to 4. Here, FIG. 2 schematically shows a first x-ray image 1 that has been recorded with a first set of recording parameters optimized for a stent 2 as the selected object class. The stent, made of iron or steel in this case, is clearly visible including its fine-meshed structure. In the recording area, however, there is a coil 3 as a further selected object, made of significantly more weakening platinum and whose structure when using the first set of recording parameters without resolution ultimately has a “spotty” appearance.

FIG. 3 shows a second x-ray image 4 that has been recorded with a second set of recording parameters matched to the coil 3. The stent 2 is hardly identifiable in this x-ray image 4, however the structure of the coil 3 is correctly resolved.

FIG. 4 shows the output image 5 determined from the x-ray images 1 and 4, in which both the stent 2 with its structure and the coil 3 with its structure are clearly visible and identifiable. For example, the output image 5 may be generated by segmenting the coil 3 in x-ray image 1 and replacing it with the x-ray image data of the coil 3 from x-ray image 4. However, as described above, other approaches to generating the output image 5 in act S3 are possible.

When generating the output image for some or all selected objects, in particular those that may be difficult to represent well in the x-ray imaging, for example, as they are noisy, an improvement may be made by using, for such an object to be highlighted, image information of the object in past x-ray images, in order to determine object appearance information, in particular through statistical averaging using the current x-ray image as well, and modifying a current x-ray image 1, 4, in particular recorded with the set of recording parameters assigned to the object to be highlighted, and/or the current output image 5 using the object appearance information. Here, in particular, the object appearance information in an image area of the object to be highlighted may be used instead of the current image information. In this way, an object-specific improvement in the representation/visibility based on time averaging of the image information is achieved, by extracting and enhancing the object details and reducing statistical noise. This improvement may also be achieved for a plurality of selected objects, in particular simultaneously.

In act S4, a check is made that an update condition has been met. Here, it may be provided that an updating of the known objects, the priority values and the selected objects takes place at regular intervals, for example, after every nth x-ray image 1, 4, or every nth output image 5. It is also conceivable however, to check the update condition as to whether, in addition or alternatively a certain event has taken place, e.g., to provide for an event-based updating. The occurrence of a user-defined trigger may be used as an event.

In certain examples, the occurrence of the event may be determined using the dynamic information and the workflow information. If for another object, for example, the highest dynamics occur, this indicates that this object is now more relevant. In the same way, the occurrence in a new workflow act may mean that the relevance of objects is changing. In all such cases, it is expedient to update the list of known objects and their priority value and to check the selected objects accordingly and change them as necessary.

Also conceivable is checking as an event, the presence of an external trigger signal, for example, from an active substance injector and/or a robot device. Such external information may also be included in the determination of the workflow information and/or the dynamic information.

If the update condition is met, act S1 is continued with. If the update condition is not met, the recording process is continued. In act S4, an abort condition may also be checked for, and where this occurs, the image acquisition and thus the monitoring of the process is ended. The abort condition may evaluate a user input, but may also be automatically monitored, for example, in turn using the workflow information.

FIG. 5 shows a schematic diagram of an x-ray device 6. The x-ray device 6 includes a C-arm 7 that may be movably mounted on a stand 8. On the C-arm 7, an x-ray emitter arrangement 9 and an x-ray detector 10 are arranged opposite one another. The x-ray emitter arrangement 9 includes in this case an x-ray tube 11 and a filter arrangement 12 with various, exchangeable filter elements. By the x-ray emitter arrangement 9, an x-ray field 13 may be generated, the x-rays from which are received and measured by the x-ray detector 10. An object under examination, (e.g., a patient), may be positioned by a patient table not shown in further detail here, in the x-ray field 13, in such a way that the recording area 14 may be captured. For the sake of clarity, objects present in the recording area 14 are not shown in more detail.

The raw data captured by the x-ray detector 10 is processed by the image generation chain 15 to create x-ray images 1, 4.

Operation of the x-ray device 6 is controlled by a control device 16, which is in particular also designed for performing the method as described herein. The control device 16 is also connected to an output device 17, (e.g., a monitor), on which the x-ray images 1, 4 or output images 5 may be displayed.

The x-ray device 6 may be part of a medical intervention workplace, for example, an angiography environment. Where patients are the object under examination the monitoring takes place by the x-ray images, in particular fluoroscopically. The other components not further represented here include, for example, sensors, an injector, a robot device and the like.

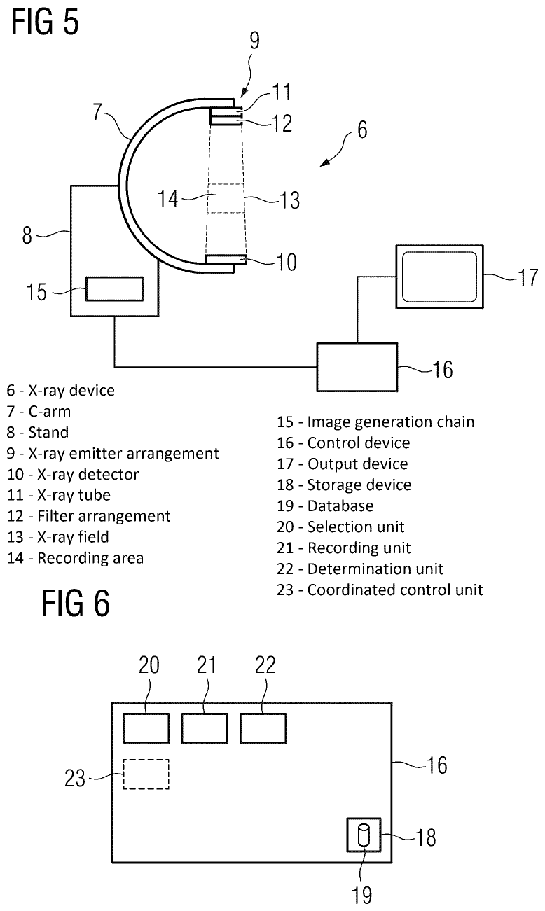

FIG. 6 shows the functional structure of the control device 16 in more detail. This includes to begin with a storage device 18, in which, for example, the database 19 with the sets of recording parameters assigned to the object classes may be stored. Other information, in particular image data, may also be stored there.

By a selection unit 20, as described in act S1, the list of known objects with the priority values is managed and objects are selected that are to be represented in a clearly visible, in particular optimized manner. In a recording unit 21, the recording of x-ray images 1, 4 then takes place according to act S2 by controlling the x-ray emitter arrangement 9, of the x-ray detector 10 and the image generation chain 15. In a determination unit 22, in particular with two selected objects, from each pair of x-ray images 1, 4, according to act S3 a determination and output of output images 5 may take place.

A superordinate control of the implementation of the method described here may take place by the selection unit 20, but optionally a coordinated control unit 23 may be provided which then in particular may also monitor the update condition and the abort condition according to act S4.

Other functional units not represented in more detail here may be provided, for example other determination units for determination of the workflow information and the dynamic information and/or the control device 16 may include at least one interface for receiving external triggers, in particular with regard to the update condition and/or the abort condition.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for operating an x-ray device having an x-ray emitter arrangement, an x-ray detector for receiving x-ray radiation of an x-ray radiation field emitted by the x-ray emitter arrangement, an image generation chain for determining an x-ray image of a recording area from raw data recorded with the x-ray detector, and an output device for outputting an output image comprising or determined from the x-ray image, wherein the method comprises:
recording x-ray images of the recording area; and
determining output images to be output from the x-ray images,
wherein, when the x-ray images are recorded, there is a repeated change between at least two sets of recording parameters that leads to a different representation of at least two different objects of the recording area in the x-ray images, and
wherein each output image of the output images is determined at least from one set of x-ray images that comprises x-ray images recorded with two or more sets of recording parameters of the at least two sets of recording parameters.

2. The method of claim 1, wherein the recording of the x-ray images is for monitoring the recording area.

3. The method of claim 1, wherein the at least two sets of recording parameters differ in pairs in at least one recording parameter of the x-ray emitter arrangement and/or in at least one recording parameter of the image generation chain.

4. The method of claim 3, wherein the at least one recording parameter of the x-ray emitter arrangement is selected from a tube voltage of an x-ray tube of the x-ray emitter arrangement, a tube current of the x-ray tube, a pulse width of the x-ray tube, a filter element of the x-ray emitter arrangement to be used, a focus size of a focal point of the x-ray emitter arrangement, a pulse length of an x-ray pulse, or a combination thereof, and/or
wherein the at least one recording parameter of the image generation chain is selected from a noise treatment parameter, a filtering parameter, a resolution parameter, a correction parameter, or a combination thereof.

5. The method of claim 4, wherein the at least one recording parameter of the image generation chain relates to an artifact correction, a movement correction, or a combination thereof.

6. The method of claim 1, wherein each set of recording parameters of the at least two sets of recording parameters is used varyingly for one x-ray image according to a predetermined sequence, alternating in a case of two sets of recording parameters.

7. The method of claim 6, wherein a frequency of changing the sets of recording parameters of the at least two sets of recording parameters is higher than a frequency for determining the output images to be output.

8. The method of claim 7, wherein the frequency of changing the sets of recording parameters comprises recording exactly one x-ray image for each set of recording parameters for determining each output image of the output images.

9. The method of claim 1, wherein, for the determination of the output image from the x-ray images of different sets of recording parameters:
at least partial overlaying of at least two x-ray images of the x-ray images takes place; and/or
x-ray image data of at least one x-ray image in a region, determined by segmenting an object in a same x-ray image or another x-ray image of the x-ray images, is replaced by x-ray image data of another x-ray image of the x-ray images and/or replacement image data representing the segmented object; and/or
the output image has at least a partial pixel- and/or patch-based composition.

10. The method of claim 9, wherein the at least partial overlaying is a weighted overlaying of the at least two x-ray images.

11. The method of claim 1, wherein each set of recording parameters is assigned to an object class of a selected object present in the recording area and is configured to the object class, and
wherein a visibility of objects of the object class is higher in x-ray images recorded with a set of recording parameters associated with the object class than in x-ray images recorded with sets of recording parameters associated with other object classes.

12. The method of claim 11, wherein the objects are selected at least partially automatically based on a prioritization of known objects, during which all known objects are given a priority value.

13. The method of claim 12, wherein the determination of the priority value of the prioritization for at least one known object is carried out taking into account dynamic information that describes a current dynamic of the at least one known object.

14. The method of claim 13, wherein, in a determination of the dynamic information, at least one x-ray image showing the respective known object is evaluated by a dynamic determination function.

15. The method of claim 14, wherein the at least one x-ray image is a time series of x-ray images, and
wherein the dynamic determination function is a trained dynamic determination function.

16. The method of claim 12, wherein the determination of the priority value of the prioritization for at least one known object takes place taking workflow information relating to a monitored process into account.

17. The method of claim 12, wherein an update of known objects and/or priority values of the selected objects takes place at regular intervals after each nth x-ray image and/or output image, or the update takes place on an event basis.

18. An x-ray device comprising:
an x-ray emitter arrangement;
an x-ray detector configured to receive x-ray radiation of an x-ray radiation field emitted by the x-ray emitter arrangement;
an image generation chain configured to determine an x-ray image of a recording area from raw data recorded with the x-ray detector;
an output device configured to output an output image comprising or determined from the x-ray image; and
a control device configured to:
record a series of x-ray images of the recording area; and
determine a series of output images to be output from the x-ray images,
wherein, when the x-ray images are recorded, the control device is configured to repeatedly change between at least two sets of recording parameters that leads to a different representation of at least two different objects of the recording area in the x-ray images, and determine each output image of the output images from at least from one set of x-ray images that comprises x-ray images recorded with two or more sets of recording parameters of the at least two sets of recording parameters.

19. A computer program or an electronically readable data carrier comprising the computer program, wherein the computer program comprises program code such that the computer program, when executed on a control device of an x-ray device, causes the control device to:
record x-ray images of a recording area; and
determine output images to be output from the x-ray images,
wherein, when the x-ray images are recorded, there is a repeated change between at least two sets of recording parameters that leads to a different representation of at least two different objects of the recording area in the x-ray images, and
wherein each output image of the output images is determined at least from one set of x-ray images that comprises x-ray images recorded with two or more sets of recording parameters of the at least two sets of recording parameters.
